(19) United States
(12) Patent Application Publication
(10) Pub. No.: US 2024/0189343 A1
(43) Pub. Date: Jun. 13, 2024

(54) TREATMENT OF VIRUS INFECTIONS

(71) Applicant: TX MEDIC AB, Viken (SE)

(72) Inventor: Lars BRUCE, Viken (SE)

(21) Appl. No.: 18/554,006

(22) PCT Filed: Jun. 10, 2022
(86) PCT No.: PCT/SE2022/050577

§ 371 Date: Oct. 5, 2023

(30) Foreign Application Priority Data
Apr. 14, 2021 (WO) PCT/SE2021/050344
Oct. 14, 2021 (SE) 2151258-7
Dec. 21, 2021 (SE) 2151582-0

(51) Int. Cl. A61K 31/737; A61P 31/14

(52) U.S. Cl. CPC A61K 31/737; A61P 31/14

(57) ABSTRACT

The invention relates to dextran sulfate, or a pharmaceutically acceptable salt thereof, for use in prevention, inhibition and/or treatment of a flavivirus infection or infectious disease. The dextran sulfate, or the pharmaceutically acceptable salt thereof, has an average molecular weight equal to or below 10000 Da and an average sulfur content of equal to or above 15%.

### TECHNICAL FIELD

The present invention generally relates to treatment of virus infections and infectious diseases, and in particular to the use of dextran sulfate in treatment of flavivirus infections and infectious diseases caused by flavivirus infections.

### BACKGROUND

Flavivirus is a genus of positive-strand ribonucleic acid (RNA) viruses in the family Flaviviridae. The genus includes the West Nile virus, dengue virus, tick-borne encephalitis virus, yellow fever virus, Zika virus and several other viruses which may cause encephalitis.

Flaviviruses generally have several common characteristics, including size (40-65 nm), symmetry (enveloped, icosahedral nucleocapsid), nucleic acid (positive-sense, single-stranded RNA around 10,000-11,000 bases), and appearance under the electron microscope.

Most of these viruses are primarily transmitted by the bite from an infected arthropod (mosquito or tick), and, hence, are classified as arboviruses. Human infections with most of these arboviruses are incidental, as humans are unable to replicate the virus to high enough titers to re-infect the arthropods needed to continue the virus lifecycle—humans are then a dead end host. The exceptions to this are the yellow fever virus, dengue virus and Zika virus. These three viruses still require mosquito vectors but are well-enough adapted to humans as to not necessarily depend upon animal hosts.

Flaviviruses may cause various infectious diseases depending on the particular viral species infecting a human host. Dengue viruses cause dengue fever, which in severe cases may develop into dengue hemorrhagic fever or dengue shock syndrome commonly referred to as severe dengue that is potentially lethal. Zika viruses cause Zika fever, also known as Zika virus disease, which have symptoms similar to dengue fever. However, mother-to-child transmission during pregnancy can cause microcephaly and other bran malformations in babies. Furthermore, Zika virus infections have been linked to Guillain-Barre syndrome (GBS) in adults. Yellow fever is caused by yellow fever virus and has symptoms, including fever, chills, loss of appetite, nausea, muscle pains, which often passes within five days. However, about 15% of the infected people develop liver damage causing yellowish skin and kidney problems.

U.S. 2004/0009953 and WO 2005/004882 investigated the effect the antiviral effect of high molecular weight (40 kDa, 500 kDa) and low sulfated (6-13%) dextran sulfate against HIV-1 virus. Antiviral Research 2017, 143: 186-194 investigated the effects of highly sulfated heparin, high molecular weight and low sulfated dextran sulfate (2.3 sulfur per disaccharide, 40 kDa) and suramin against Zika virus infection.

There is a general need for treatments that reduce the risk of infection and spread of the virus once a subject has been infected with flaviviruses.

### SUMMARY

It is a general objective to provide a treatment of flavivirus infections or infectious diseases.

This and other objectives are met by embodiments as disclosed herein.

An aspect of the invention relates to dextran sulfate, or a pharmaceutically acceptable salt thereof, for use in prevention, inhibition and/or treatment of a flavivirus infection or infectious disease. The dextran sulfate, or the pharmaceutically acceptable salt thereof, has an average molecular weight equal to or below 10 000 Da and an average sulfur content of equal to or above 15%.

Experimental data as presented herein indicates that dextran sulfate, or a pharmaceutically acceptable salt thereof, may be used in treating flavivirus infections and flavivirus infectious diseases. In more detail, dextran sulfate caused a significant and dose dependent inhibition of Zika virus, dengue virus and yellow fever virus infection of human cells at clinically relevant concentrations.

### BRIEF DESCRIPTION OF THE DRAWINGS

The embodiments, together with further objects and advantages thereof, may best be understood by making reference to the following description taken together with the accompanying drawings, in which:

FIG. 1 illustrates that dextran sulfate inhibits Zika virus infection in human Hela cells in a concentration dependent manner at MOI 0.5 (1A), 3 (1B) and 5 (1C). Data represent the mean+SEM.

FIG. 2 illustrates that dextran sulfate inhibits Zika virus infection in Vero cells in a concentration dependent manner at MOI 0.05 (2A) and 0.01 (2B). Data represent the mean+SEM.

FIG. 3 illustrates control monensin inhibition of Zika virus infection in human Hela cells.

FIGS. 4A-4H illustrate the ability of dextran sulfate and the positive control virus infectivity inhibitor monensin to prevent infection of mammalian cells by the indicated flaviviruses in vitro. Concentration-effect data were analyzed by iterative curve fitting according to a four parameter Logistic equation. UM on the x-axis for dextran sulfate and monensin, respectively. Data represent individual data points from a single experiment.

FIG. 5 illustrates that dextran sulfate inhibits>90% dengue virus, Zika virus and yellow fever virus infection into human cells in a concentration-dependent manner. Data represent the mean+SD.

### DETAILED DESCRIPTION

The present invention generally relates to treatment of virus infections and infectious diseases, and in particular to the use of dextran sulfate in treatment of flavivirus infections and infectious diseases caused by flavivirus infections.

Dextran sulfate was capable of preventing flavivirus infection, as represented by Zika virus (ZIKV), dengue virus (DENV) and yellow fever virus (YFV) infection, in human cells at clinically relevant concentrations. In more details, dextran sulfate treatment significantly reduced the number of infected cells as compared to control providing evidence that dextran sulfate is capable of significantly preventing or at least inhibiting flavivirus infection in human cells.

Flavivirus particles or virions are enveloped and spherical with a diameter of about 50 nm. The surface proteins are arranged in an icosahedral-like symmetry. Mature virions mainly contain two virus-encoded membrane proteins, denoted protein M and protein E, while immature virions contain a membrane protein precursor.

Flavivirus infection in general and Zika, dengue and yellow fever virus infection in particular could be regarded as a two-step process with an initial virus-to-cell attachment followed by internalization into the host cell. The initial virus-to-cell attachment is mediated via interactions between viral envelope protein E and negatively charged glycosaminoglycans (GAGs) on the cellular surface (Infection, Genetics and Evolution 2019 69: 22-29) and enriches virus particles on the cell surface. The actual internalization into the host cell is mediated by cell surface receptors of the host cell. Neural cell adhesion molecule (NCAM1) has recently been identified as a potential flavivirus receptor, and in particular Zika virus receptor, for cell internalization (Nature Communications 2020 11: 3896) as has heparan sulfate (Virology 2002, 292: 162-168). However, no direct Zika virus-heparin interaction was observed in heparin-binding analysis and downregulation or removal of cellular heparan sulfate did not inhibit Zika virus infection (Antiviral Research 2017, 143: 186-194). This indicated that cell surface heparan sulfate is not utilized by Zika virus as an attachment receptor.

Possible mechanisms for the inhibitory effects of dextran sulfate of flavivirus infection may be to interfere with the initial virus-to-cell attachment and/or to the binding to cell surface receptors. Dextran suflate of the embodiments present negative charges and may mimic heparan sulfate and other negatively charged GAGs on cellular surfaces. Accordingly, dextran sulfate of the embodiments could block or at least interfere with the binding between viral envelope protein E and heparan sulfate and other negatively charged GAGs on the surface of host cells. In addition, NCAM1 and other cell surface receptors include heparin binding domains (HBDs) (Journal of Neuroscience Research 1992 33(4): 538-548). Dextran sulfate of the embodiments is capable of binding to such HBDs or acting as a competitive antagonist and may, thus, interfere with the binding of flavivirus particles to NCAM1 and other cell surface receptors comprising such HBDs or heparan sulfate.

The experimental results as seen with dextran sulfate of the embodiments may, thus, be obtained by interfering with the initial virus-to-cell attachment and interfering with the interaction between virus proteins and cell surface receptors, such as NCAM1 and heparan sulfate.

An aspect of the invention relates to dextran sulfate, or a pharmaceutically acceptable salt thereof, for use in preventing, inhibiting or treating a flavivirus infection or infectious disease.

In an embodiment, the flavivirus infection is selected from the group consisting of a flavivirus infection caused by a flavivirus selected from the group consisting of dengue virus (DENV), Japanese encephalitis virus (JEV), Kunjin virus (KUNV), Langat virus (LGTV), Louping ill virus (LIV), Murray valley encephalitis virus (MVEV), St. louis encephalitis virus (SLEV), powassan virus (POWV), West Nile virus (WNV), Yellow fever virus (YFV) and Zika virus (ZIKAV). In another embodiment, the flavivirus infection is selected from the group consisting of a flavivirus infection caused by a flavivirus selected from the group consisting of DENV, JEV, KUNV, LIV, MVEV, SLEV, POWV, WNV, YFV and ZIKV.

In a particular embodiment, the flavivirus infection is selected from the group consisting of a flavivirus infection caused by a flavivirus selected from the group consisting of DENV, YFV and ZIKV.

In a preferred embodiment, the flavivirus infection is a flavivirus infection caused by ZIKV.

In another preferred embodiment, the flavivirus infection is a flavivirus infection caused by DENV.

In a further embodiment, the flavivirus infection is a flavivirus infection caused by YFV.

In an embodiment, the flavivirus infection is caused by a flavivirus other than Japanese encephalitis virus, West Nile virus, dengue virus, and Yellow fever virus.

Flavivirus infectious diseases as used herein include dengue fever, dengue heorrhagic fever and dengue shock syndrome caused by DENV, Japanese encephalitis caused by JEV, non-encephalitic Kunjin virus disease and encephalitic Kunjin virus disease caused by KUNV, Louping-ill caused by LIV, Murray valley encephalitis caused by MVEV, St. louis encephalitis caused by SLEV, encephalitis caused by POWV, West Nile fever caused by WNV, yellow fever caused by YFV, and Zika fever or Zika virus disease caused by ZIKAV.

In a preferred embodiment, the flavivirus infectious disease is selected from the group consisting of dengue fever, dengue heamorrhagic fever and dengue shock syndrome caused by DENV, yellow fever caused by YFV, and Zika fever or Zika virus disease caused by ZIKAV. In a particular embodiment, the flavivirus infectious disease is Zika fever or Zika virus disease caused by ZIKAV. In another particular embodiment, the flavivirus infectious disease is dengue fever, dengue heamorrhagic fever or dengue shock syndrome caused by DENV. In a further particular embodiment, the flavivirus infectious disease is yellow fever caused by YFV.

In an embodiment, the flavivirus infectious disease is a flavivirus infectious disease other than Japanese encephalitis caused by JEV, West Nile fever caused by WNV, dengue fever, dengue heorrhagic fever and dengue shock syndrome caused by DENV and yellow fever caused by YFV.

The present invention also relates to use of dextran sulfate, or a pharmaceutically acceptable salt thereof, for the manufacture of a medicament for the prevention, inhibition and/or treatment of a flavivirus infection or infectious disease.

The present invention further relates to a method for preventing, inhibiting and/or treating a flavivirus infection or infectious disease. The method comprises administering an effective amount of dextran sulfate, or a pharmaceutically acceptable salt thereof, to a subject suffering from the flavivirus infection or infectious disease or having a risk of suffering from the flavivirus infection or infectious disease.

Treatment of a flavivirus infection or infectious disease as used herein does not necessarily mean curative treatment of the flavivirus infection or infectious disease but also encompass inhibition or reduction of the short- and long-term symptoms of the flavivirus infection or infectious disease. Hence, treatment also encompass delaying onset of the flavivirus infection or infectious disease, including delaying, preventing onset of symptoms or resolving established pathologies associated with the flavivirus infection or infectious disease.

Also other viruses use a similar mechanism as flavivirus in attaching to and internalizing cells. For instance, the rabies virus is also using the cell surface receptor NCAM1 for internalization (Philosophical Transactions Royal Society B 2015, 370: 2014035). In more detail, rabies viruses and other lyssaviruses comprises a glycoprotein G that is involved in the interaction with the host cell and internalization. Hence, dextran sulfate of the embodiments could also be used in preventing, inhibiting or treating a lyssavirus infection or infectious disease.

In an embodiment, the lyssavirus infection is selected from the group consisting of a lyssavirus infection caused by a lyssavirus selected from the group consisting of Australian bat lyssavirus (ABLV), Duvenhage lyssavirus (DUVV), European bat lyssavirus (EBLV), Lagos bat virus (LBV), Mokola virus (MOKV), and rabies virus. In a particular embodiment, the lyssavirus infection is caused by rabies virus.

In an embodiment, the lyssavirus infectious disease is rabies, encephalitis or a rabies-like encephalitic illness. In a particular embodiment, the lyssavirus infectious disease is rabies.

In an embodiment, the lyssavirus infection is caused by a lyssavirus other than rabies virus.

In the following, reference to (average) molecular weight and sulfur content of dextran sulfate applies also to any pharmaceutically acceptable salt of dextran sulfate. Hence, the pharmaceutically acceptable salt of dextran sulfate preferably has the average molecular weight and sulfur content as discussed in the following embodiments.

Dextran sulfate outside of the preferred ranges of the embodiments are believed to have inferior effect and/or causing negative side effects to the cells or subject.

For instance, dextran sulfate of a molecular weight exceeding 10,000 Da (10 kDa) generally has a lower effect vs. side effect profile as compared to dextran sulfate having a lower average molecular weight. This means that the maximum dose of dextran sulfate that can be safely administered to a subject is lower for larger dextran sulfate molecules (>10,000 Da) as compared to dextran sulfate molecules having an average molecular weight within the preferred ranges. As a consequence, such larger dextran sulfate molecules are less appropriate in clinical uses when the dextran sulfate is to be administered to subjects in vivo. For instance, U.S. 2004/0009953 discloses that dextran sulfate having an average molecular weight of 40 kDa or 500 kDa and an average sulfur content of 17% was highly toxic to rats.

Dextran sulfate, or the pharmaceutically acceptable salt thereof, according to the invention is not toxic as shown in the presented Examples. Hence, the dextran sulfate, or the pharmaceutically acceptable salt thereof, of the invention do not have the toxicity problems as compared to the high molecular weight dextran sulfate as disclosed in U.S. 2004/0009953.

Dextran sulfate is a sulfated polysaccharide and in particular a sulfated glucan, i.e., polysaccharide made of many glucose molecules. Average molecular weight as defined herein indicates that individual sulfated polysaccharides may have a molecular weight different from this average molecular weight but that the average molecular weight represents the mean molecular weight of the sulfated polysaccharides. This further implies that there will be a natural distribution of molecular weights around this average molecular weight for a dextran sulfate sample.

Average molecular weight, or more correctly weight average molecular weight (M_{w}), of dextran sulfate is typically determined using indirect methods such as gel exclusion/penetration chromatography, light scattering or viscosity. Determination of average molecular weight using such indirect methods will depend on a number of factors, including choice of column and eluent, flow rate, calibration procedures, etc.

Weight average molecular weight (M_{w}):

$$\frac{\sum {M}_{i}^{2} {N}_{i}}{\sum {M}_{i} {N}_{i}} ,$$

typical for methods sensitive to molecular size rather than numerical value, e.g., light scattering and size exclusion chromatography (SEC) methods. If a normal distribution is assumed, then a same weight on each side of M_{w}, i.e., the total weight of dextran sulfate molecules in the sample having a molecular weight below M_{w} is equal to the total weight of dextran sulfate molecules in the sample having a molecular weight above M_{w}. The parameter Ni indicates the number of dextran sulfate molecules having a molecular weight of Mᵢ in a sample or batch.

In an embodiment, the dextran sulfate, or the pharmaceutically acceptable salt thereof, has a M_{w} equal to or below 10,000 Da. In a particular embodiment, the dextran sulfate, or the pharmaceutically acceptable salt thereof, has a M_{w} within an interval of from 2,000 Da to 10,000 Da.

In another embodiment, the dextran sulfate, or the pharmaceutically acceptable salt thereof, has a M_{w} within an interval of from 2,500 Da to 10,000 Da, preferably within an interval of from 3,000 Da to 10,000 Da. In a particular embodiment, the dextran sulfate, or the pharmaceutically acceptable salt thereof, has a M_{w} within an interval of from 3,500 Da to 9,500 Da, such as within an interval of from 3,500 Da to 8,000 Da.

In another particular embodiment, the dextran sulfate, or the pharmaceutically acceptable salt thereof, has a M_{w} within an interval of from 4,500 Da to 7,500 Da, such as within an interval of from 4,500 Da and 6,500 Da or within an interval of from 4,500 Da and 5,500 Da.

Thus, in some embodiments, the dextran sulfate, or the pharmaceutically acceptable salt thereof, has a M_{w} equal to or below 10,000 Da, equal to or below 9,500 Da, equal to or below 9,000 Da, equal to or below 8,500 Da, equal to or below 8,000 Da, equal to or below 7,500 Da, equal to or below 7,000 Da, equal to or below 6,500 Da, equal to or below 6,000 Da, or equal to or below 5,500 Da.

In some embodiments, the dextran sulfate, or the pharmaceutically acceptable salt thereof, has a M_{w} equal to or above 1,000 Da, equal to or above 1,500 Da, equal to or above 2,000 Da, equal to or above 2,500 Da, equal to or above 3,000 Da, equal to or above 3,500 Da, equal to or above 4,000 Da. or equal to or above 4,500 Da. Any of these embodiments may be combined with any of the above presented embodiments defining upper limits of the M_{w}, such combined with the upper limit of equal to or below 10,000 Da.

In a particular embodiment, the M_{w} of dextran sulfate, or the pharmaceutically acceptable salt thereof, as presented above is average M_{w}, and preferably determined by gel exclusion/penetration chromatography, size exclusion chromatography, light scattering or viscosity-based methods.

Number average molecular weight (Mₙ):

$$\frac{\sum {M}_{i} {N}_{i}}{\sum {N}_{i}} ,$$

typically derived by end group assays, e.g., nuclear magnetic resonance (NMR) spectroscopy or chromatography. If a normal distribution is assumed, then a same number of dextran sulfate molecules can be found on each side of Mₙ, i.e., the number of dextran sulfate molecules in the sample having a molecular weight below Mₙ is equal to the number of dextran sulfate molecules in the sample having a molecular weight above Mₙ.

In an embodiment, the dextran sulfate, or the pharmaceutically acceptable salt thereof, has a Mₙ as measured by NMR spectroscopy within an interval of from 1,850 to 3,500 Da.

In a particular embodiment, the dextran sulfate, or the pharmaceutically acceptable salt thereof, has a Mₙ as measured by NMR spectroscopy within an interval of from 1,850 Da to 2,500 Da, preferably within an interval of from 1,850 Da to 2,300 Da, such as within an interval of from 1,850 Da to 2,000 Da.

Thus, in some embodiments, the dextran sulfate, or the pharmaceutically acceptable salt thereof, has a Mₙ equal to or below 3,500 Da, equal to or below 3,250 Da, equal to or below 3,000 Da, equal to or below 2,750 Da, equal to or below 2,500 Da, equal to or below 2,250 Da, or equal to or below 2,000 Da. In addition, the dextran sulfate, or the pharmaceutically acceptable salt thereof, has a Mₙ equal to or above 1,850 Da.

In an embodiment, the dextran sulfate, or the pharmaceutically acceptable salt thereof, has an average sulfate number per glucose unit within an interval of from 2.5 to 3.0.

In a particular embodiment, the dextran sulfate, or the pharmaceutically acceptable salt thereof, has an average sulfate number per glucose unit within an interval of from 2.5 to 2.8, preferably within an interval of from 2.6 to 2.7.

In an embodiment, the dextran sulfate, or the pharmaceutically acceptable salt thereof, has an average number of glucose units within an interval of from 4.0 to 6.0.

In a particular embodiment, the dextran sulfate, or the pharmaceutically acceptable salt thereof, has an average number of glucose units within an interval of from 4.5 to 5.5, preferably within an interval of from 5.0 to 5.2.

In an embodiment, the dextran sulfate, or the pharmaceutically acceptable salt thereof, has a Mₙ as measured by NMR spectroscopy within an interval of from 1,850 to 3,500 Da, an average sulfate number per glucose unit within an interval of from 2.5 to 3.0, and an average sulfation of C2 position in the glucose units of the dextran sulfate is at least 90%.

In an embodiment, the dextran sulfate has an average number of glucose units of about 5.1, an average sulfate number per glucose unit within an interval of from 2.6 to 2.7 and a Mₙ within an interval of from 1,850 Da and 2,000 Da.

In an embodiment, the pharmaceutically acceptable salt of dextran sulfate is a sodium salt of dextran sulfate. In a particular embodiment, the sodium salt of dextran sulfate has an average number of glucose units of about 5.1, an average sulfate number per glucose unit within an interval of from 2.6 to 2.7 and a Mₙ including the Na+counter ion within an interval of from 2,100 Da to 2,300 Da.

In an embodiment, the dextran sulfate has an average number of glucose units of 5.1, an average sulfate number per glucose unit of 2.7, an average Mₙ without Na+ as measured by NMR spectroscopy of about 1,900-1,950 Da and an average Mₙ with Na+ as measured by NMR spectroscopy of about 2,200-2,250 Da.

Dextran sulfate is a polyanionic derivate of dextran and contains sulfur. The average sulfur content for dextran sulfate of the embodiments is equal to or more than 15%. In an embodiment, the average sulfur content for dextran sulfate of the embodiments is preferably from 15 to 20% and more preferably approximately 17%, generally corresponding to about or at least two sulfate groups per glucosyl residue. In a particular embodiment, the sulfur content of dextran sulfate is preferably equal to or at least close to the maximum possible degree of sulfur content of the corresponding dextran molecules.

The dextran sulfate according to the embodiments can be provided as a pharmaceutically acceptable salt of dextran sulfate, such as a sodium or potassium salt.

A currently preferred dextran sulfate according to the embodiments is disclosed in WO 2016/076780.

The subject is preferably a mammalian subject, more preferably a primate and in particular a human subject. The dextran sulfate, or the pharmaceutically acceptable salt thereof, can, however, be used also in veterinary applications. Non-limiting example of animal subjects include primate, cat, dog, pig, horse, mouse, rat.

The dextran sulfate, or the pharmaceutically acceptable salt thereof, is preferably administered by injection to the subject and in particular by intravenous (i.v.) injection, subcutaneous (s.c.) injection or (i.p.) intraperitoneal injection, preferably i.v. or s.c. injection. Other parenteral administration routes that can be used include intramuscular and intraarticular injection. Injection of the dextran sulfate, or the pharmaceutically acceptable derivative thereof, could alternatively, or in addition, take place directly in, for instance, a tissue or organ or other site in the subject body, at which the target effects are to take place.

The dextran sulfate, or the pharmaceutically acceptable salt thereof, of the embodiments is preferably formulated as an aqueous injection solution with a selected solvent or excipient. The solvent is advantageously an aqueous solvent and in particular a buffer solution. A non-limiting example of such a buffer solution is a citric acid buffer, such as citric acid monohydrate (CAM) buffer, or a phosphate buffer. For instance, dextran sulfate of the embodiments can be dissolved in saline, such as 0.9% NaCl saline, and then optionally buffered with 75 mM CAM and adjusting the pH to about 5.9 using sodium hydroxide. Also, non-buffered solutions are possible, including aqueous injection solutions, such as saline, i.e., NaCl (aq). Furthermore, other buffer systems than CAM could be used if a buffered solution are desired.

The embodiments are not limited to injections and other administration routes can alternatively be used including orally, nasally, bucally, rectally, dermally, tracheally, bronchially, or topically. The active compound, dextran sulfate, is then formulated with a suitable excipient or carrier that is selected based on the particular administration route.

Suitable dose ranges for the dextran sulfate, or the pharmaceutically acceptable salt thereof, may vary according to the application, such as in vitro versus in vivo, the size and weight of the subject, the condition for which the subject is treated, and other considerations. In particular for human subjects, a possible dosage range could be from 1 μg/kg to 100 mg/kg of body weight, preferably from 10 μg/kg to 50 mg/kg of body weight.

In preferred embodiments, the dextran sulfate, or the pharmaceutically acceptable salt thereof, is formulated to be administered at a dosage in a range from 0.05 to 50 mg/kg of body weight of the subject, preferably from 0.05 or 0.1 to 40 mg/kg of body weight of the subject, and more preferably from 0.05 or 0.1 to 30 mg/kg, or 0.1 to 25 mg/kg or from 0.1 to 15 mg/kg or 0.1 to 10 mg/kg body weight of the subject. Preferred dosages are selected in a range from 0.25 to 5 mg/kg, preferably 0.5 to 2.5 mg/kg, and more preferably 0.75 to 2 mg/kg body weight of the subject.

The dextran sulfate, or the pharmaceutically acceptable derivative thereof, can be administered at a single administration occasion, such as in the form of a single bolus injection. This bolus dose can be injected quite quickly to the subject but is advantageously infused over time so that the dextran sulfate solution is infused over a few minutes of time to the patient, such as during 5 to 10 minutes.

Alternatively, the dextran sulfate, or the pharmaceutically acceptable salt thereof, can be administered at multiple, i.e., at least two, occasions during a treatment period.

The dextran sulfate, or the pharmaceutically acceptable salt thereof, can be administered together with other active agents, either sequentially, simultaneously or in the form of a composition comprising the dextran sulfate, or the pharmaceutically acceptable salt thereof, and at least one other active agent. The at least one active agent can be selected among any agent useful in any of the above mentioned diseases, disorders or conditions.

### EXAMPLES

### EXAMPLE 1 —Antiviral Activity Against ZIKV

The aim of this study was to investigate the antiviral activity of dextran sulfate against Zika virus (ZIKV).

### Materials & Methods

In this Example, a sodium salt of dextran sulfate was used (ILB®, Tikomed AB, Viken, Sweden, WO 2016/076780). The dextran sulfate was provided in a stock solution at 100 mg/ml in 0.9% NaCl.

Viral infection was investigated in two cell systems: human HeLa Kyoto cells and African green monkey kidney cells (Vero AD cells) using Zika virus PF13. The following conditions were tested: HeLa Kyoto at multiplicity of infection (MOI), i.e., the number of virions per cell at the start of the infection, 0.5, 3 and 5, and Vero AD at MOI 0.05, and 0.01. Cytotoxicity was determined using an MTT assay on uninfected cells, treated with the same concentrations of dextran sulfate.

### Reagents

Complete media: 5% M199 for Vero AD; 10% DMEM (Gibco) supplemented with 10% FBS (Gibco) and 1X p/s (Gibco) for HeLa Kyoto.

Supplemented media: Same as above for Vera AD, and the same as above for HeLa Kyoto but with 2% FBS.

### Experimental Procedure

The antiviral activity of three dilutions of dextran sulfate was explored by pre-incubating the drug with the assay cells for 1 h before infection with the Zika virus. Virus and formulations were left on the cells for the entire 48 h duration of the experiment. The cytotoxicity of the same range of concentrations of dextran sulfate was determined by MTT assay in the same cell lines. To ensure the correct range of infectivity was reached, for Zika virus three MOI were tested in parallel.

### Cell Plating

Cells were detached and counted. Two 96 well plates were seeded per cell type, one for the antiviral assay and one for the cytotoxicity assay. Cells were seeded in 100 μl/well of complete media at a density of 8,000 cells/well. After seeding, the plates were incubated at room temperature for 5 minutes for even distribution, and then at 37° C., 5% CO₂ until the following day.

The plates contained three wells per dextran sulfate concentration (600 μg/ml, 200 μg/ml, 60 μg/ml) and MOI, nine infected control wells per MOI, 18 uninfected control wells and eight monensin control wells per MOI, see table below, in which bold represents first MOI, italics represent second MOI and underlining represents third MOI.

| | 1 | 2 | 3 | 4 | 5 | 6 | 7 | 8 | 9 | 10 | 11 | 12 |
|---|---|---|---|---|---|---|---|---|---|---|---|---|
| A | 600 | 600 | 600 | 600 | 600 | 600 | 600 | 600 | 600 | M | M | M |
| B | 200 | 200 | 200 | 200 | 200 | 200 | 200 | 200 | 200 | M | M | M |
| C | 60 | 60 | 60 | 60 | 60 | 60 | 60 | 60 | 60 | M | M | M |
| D | I | I | I | I | I | I | I | I | I | M | M | M |
| E | I | I | I | I | I | I | I | I | I | M | M | M |
| F | I | I | I | I | I | I | I | I | I | M | M | M |
| G | U | U | U | U | U | U | U | U | U | M | M | M |
| H | U | U | U | U | U | U | U | U | U | M | M | M |

600: 600 μg/ml dextran sulfate;
200: 200 μg/ml dextran sulfate;
60 μg/ml dextran sulfate
M: Monensin (Monensin sodium salt, Sigma);
I: Infected;
U: Uninfected

### Compound pre-treatment

Dextran sulfate was prepared in three different concentrations from the stock solution (100 mg/ml):
600 μg/ml: 6 μl of dextran sulfate stock into 994 μl of supplemented media;
200 μg/ml: 2 μl of dextran sulfate stock into 998 μl of supplemented media; and
60 μg/ml: 100 μl of 600 μg/ml into 900 μl of supplemented media.

2.4 μl of a 10 mM monensin stock was added to 1200 μl of supplemented media followed by mixing by vortex. 240 μl of supplemented media was added into three wells of each row of a round-bottom 96 well plate (marked M above). 360 μl of diluted monensin was added into three top wells. 120 μl of the volume in the wells in the three top wells was moved down to the three wells in the next row and so forth to get three fold dilutions of monensin per row.

Media was removed from the cells and replaced with 100 μl of prepared compounds (dextran sulfate or monensin) or media (uninfected and untreated controls) for 1 h.

Towards the end of the incubation, fresh dextran sulfate and monensin were prepared at twice the final concentration in 50 μl volume, as they will be diluted to the final concentration by an equal volume of virus or media.
1,200 μg/ml: 7.2 μl of dextran sulfate stock into 592.8 μl of supplemented media
400 μg/ml: 2.4 μl of dextran sulfate stock into 597.6 μl of supplemented media
120 μg/ml: 60 μl of 1,200 μg/ml dilution into 540 μl of supplemented media

2.4 μl of a 10 mM monensin stock was added to 600 μl of supplemented media followed by mixing by vortex. 120 μl of supplemented media was added into three wells of each row of a round-bottom 96 well plate (marked M above). 180 μl of diluted monensin was added into three top wells. 60 μl of the volume in the wells in the three top wells was moved down to the three wells in the next row and so forth to get three fold dilutions of monensin per row.

### Cytotoxicity

Dextran sulfate and monensin were added at the same time as the pre-incubation drugs and left on for the duration of the experiment. Monensin was removed after 8 h and replaced with supplemented media to reduce cytotoxicity.

### Virus Preparation and Infection

Viruses were added at the MOI according to below:
Zika MOI 0.5 (HeLa Kyoto): 27 μl of undiluted virus (1.5×10⁷ PFU/ml)+2,473 μl of supplemented media;
Zika MOI 3 (HeLa Kyoto): 160 μl of undiluted virus+2,340 μl of supplemented media;
Zika MOI 5 (HeLa Kyoto): 270 μl of undiluted virus+2,230 μl of supplemented media
Zika MOI 0.5 (Vero AD): 27 μl of undiluted virus (1.5×10⁷ PFU/ml)+2,473 μl of supplemented media
Zika MOI 0.05 (Vero AD): 2.7 μl of undiluted virus+2,500 μl of supplemented media
Zika MOI 0.01 (Vero AD): 0.6 μl of undiluted virus+2,500 μl of supplemented media

At the end of the compound pre-incubation, media were removed from the cells and replaced with 50 μl of diluted compounds (dextran sulfate or monensin) or media (untreated and uninfected controls). 50 μl of diluted virus or media (uninfected control) was immediately added. The plates were incubated at 37° C. and 5% CO₂ for 48 hours and the formulations+virus were left on the cells for the entire duration of the experiment.

### Fixation and Development

After 48 h, the plates were washed with PBS, fixed for 30 mins with 4% formaldehyde, washed again with PBS, and stored in PBS at 4° C. until staining.

The cytotoxicity plates were treated with MTT to determine cell viability.

### Infectivity Readout

Cells were immunostained with immunofluorescence staining. Briefly, any residual formaldehyde was quenched with 50 mM ammonium chloride, after which cells were permeabilised (0.1% Triton X100) and stained with an antibody recognizing flavivirus envelope protein (Millipore, MAB10216). The primary antibodies were detected with an Alexa-488 conjugate secondary antibody (Life Technologies, A11001), and nuclei were stained with Hoechst. Images were acquired on a CellInsight CX5 high content platform (Thermo Scientific) using a 10X objective, and percentage infection calculated using CellInsight CX5 software (infected cells/total cells×100).

### Cytotoxicity Readout

Cytotoxicity was detected by MTT assay. Briefly, the MTT reagent (Sigma) was added to the cells for 2 h at 37° C., 5% CO₂, after which the media was removed and the precipitate solubilized with a mixture of 1:1 isopropanol:DMSO for 20 minutes. The supernatant was transferred to a clean plate and signal read at 570 nm.

### Determination of % Inhibition

Normalized percentages of inhibition were calculated using the following formula:

$$Normalized \% inhibition = 100 \times ( 1 - \frac{\% Infection Sample - \% Infection Uninfected Control}{\% Infection Infected Control - \% Infection Uninfected Control} )$$

For the control drug, EC₅₀ values were extrapolated from the curves representing the best fit (non-linear regression analysis, variable slope) of the logarithm of compound concentration vs. the normalized percentages of inhibition, using GraphPad Prism (version 9).

### Determination of Cytotoxicity

Percentages of cytotoxicity were calculated using the following formula:

$$\% cytotoxicity = 100 - ( 100 \times \frac{Absorbance Sample}{Absorbance Control} )$$

TC₅₀ values for the control drug were extrapolated from the curves representing the best fit (non-linear regression analysis, variable slope) of the logarithm of compound concentration vs. the normalized percentages of cytotoxicity, using GraphPad Prism (version 9).

### Results

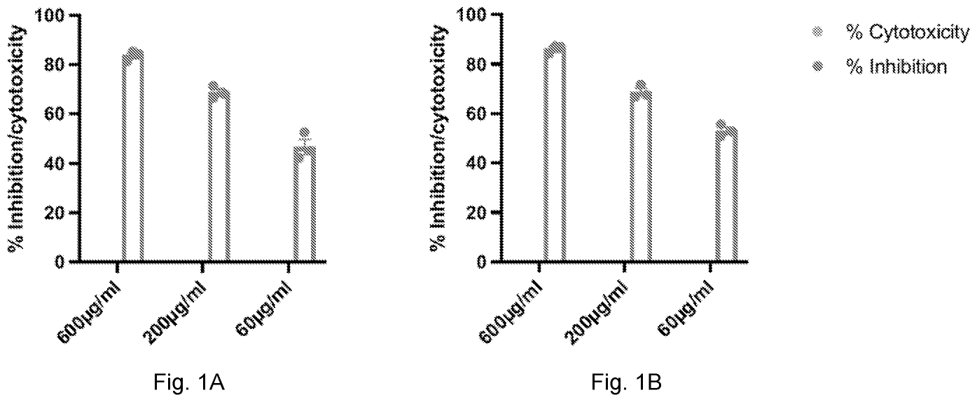

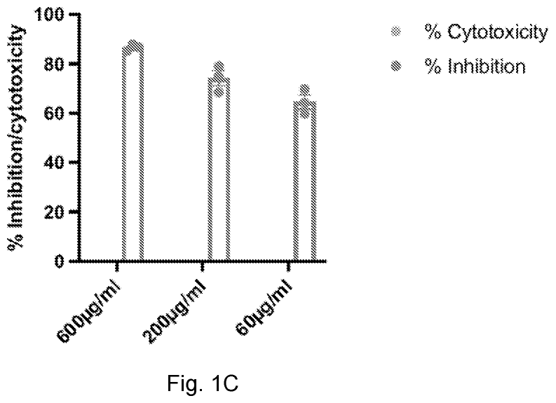

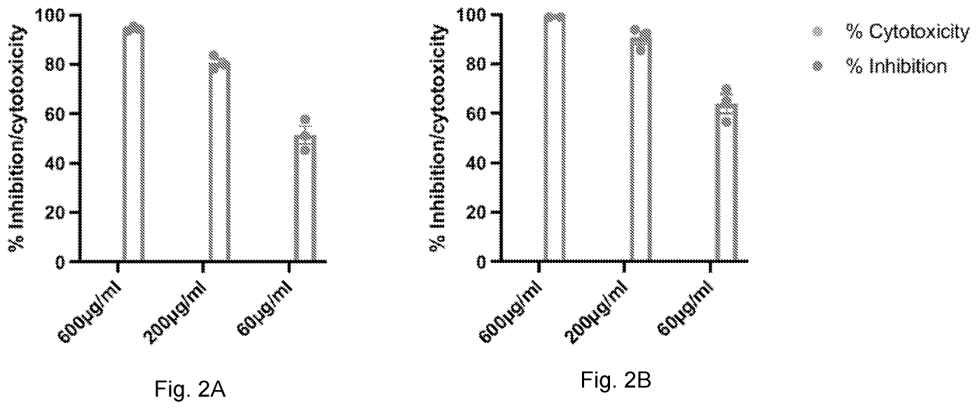

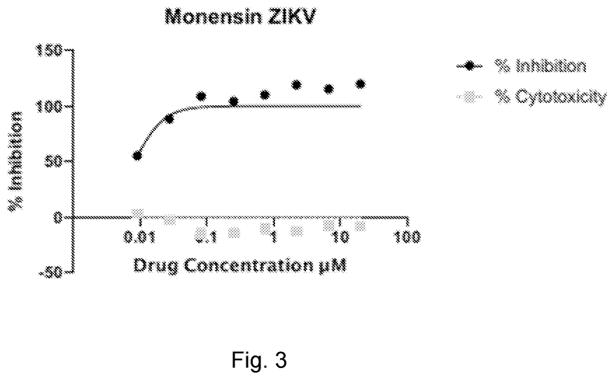

The bar charts in FIGS. 1A-1C, 2A-2B display the % of infection inhibition (bars) and the % of cytotoxicity (dots) for dextran sulfate at the three concentrations tested for the indicated viruses. Dextran sulfate caused a significant and dose dependent inhibition of Zika virus in both cell types.

No significant cytotoxicity was observed at any of the concentrations tested.

Given the unmet clinical need for effective antivirals against Zika and other flaviviruses, the results presented herein indicate that dextran sulfate of the embodiment has therapeutic potential against the flavivirus family.

The effectiveness of the dextran sulfate against Zika virus was highly surprising given that Antiviral Research 2017, 143: 186-194 showed that dextran sulfate had a % infection inhibition of about 60% at 200 μg/ml in Vero cells, as compared to a % of infection inhabitation of more than 80% at 200 μg/ml with a dextran sulfate according to the invention (FIG. 2A). The dextran sulfate used in Antiviral Research 2017, 143: 186-194 is a low sulfate dextran sulfate (2.3 sulfations per disaccharide) from Sigma. Furthermore, the dextran sulfate has a significantly higher average molecular weight (40 kDa). These differences in sulfation number and average molecular weight between the dextran sulfate of the invention and the dextran sulfate in Antiviral Research 2017, 143: 186-194 are thought to be the basis for the significant differences in antiviral activity against Zika virus.

### EXAMPLE 2 —Antiviral Activity Against DENV, ZIKV and YFV

The aim of this study was to investigate the antiviral activity of dextran sulfate against dengue virus (DENV), Zika virus (ZIKV) and yellow fever virus (YFV).

### Materials & Methods

In this Example, a sodium salt of dextran sulfate was used (ILB®, Tikomed AB, Viken, Sweden, WO 2016/076780). The dextran sulfate was provided in a stock solution at 100 mg/ml in 0.9% NaCl.

Viral infection was investigated in two cell systems: human Huh-7 cells (VRS stock P1) using a panel of four serotypes of dengue virus (DENV) and the vaccine strain of yellow fever 17D (YF17D) and human HeLa Kyoto cells (VRS stock P1) using two lineages of Zika virus (ZIKV).

Viruses: DENV1 (strain Hawaii, GenBankTM code EU848545);
DENV2 (strain New Guinea C, GenBankTM code AF038403);
DENV3 (strain H87, GenBankTM code M93130);
DENV4 (strain H241; GenBankTM code AY947539);
ZIKV African lineage (Strain MP1751);
ZIKV Asian lineage (PF/13); and
YF17D (VRS stock)

### Reagents

Complete media: DMEM (Gibco) supplemented with 10% FBS (Gibco) and 1X p/s (Gibco)
Supplemented media: DMEM (Gibco) supplemented with 2% FBS (Gibco) and 1X p/s (Gibco)

### Experimental Procedure

The antiviral activity of eight dilutions of dextran sulfate was explored by pre-incubating the drug with the assay cells for 1 h before virus addition. Virus and formulations were left on the cells for the entire 48 h duration of the experiment. The cytotoxicity of the same range of concentrations of dextran sulfate was determined by MTT assay.

### Cell Plating

Cells were detached and counted. Two 96 well plates were seeded per cell type, one for the antiviral assay and one for the cytotoxicity assay. Huh-7 and Hela cells were seeded in 100 μl/well of complete media at a density of 8,000 cells/well. After seeding, the plates were incubated at room temperature for 5 minutes for even distribution, and then at 37° C., 5% CO₂ until the following day.

### Virus Preparation and Infection

Virus stocks were diluted into supplemented media, to reach the following MOI:
DENV1-3: 0.3
DENV4: 1
ZENV: 5
YF17D: 0.1

### Formulation Dilutions

Dextran sulfate formulations were prepared at twice the final concentrations, as they become diluted to the final concentrations by an equal volume of virus or media.

For each virus, the dextran sulfate test solution was prepared by adding 150 μl of the 100 mg/ml stock to 600 μl of supplemented media (20 mg/ml).

For each relevant virus, monensin as a control inhibitor was prepared by adding 3 μl of a 10 mM stock to 747 μl of supplemented media (40 μM).

In row A of a round bottom 96-well plate, the dilutions prepared above were added in triplicate (180 μl/well). In rows B to H, 120 μl of supplemented media were added. In a 3-fold serial dilution, 60 μl were transferred from the previous row to the next down, mixing 15 times in between.

### Cell Treatment

Media was removed from the cells and 50 μl of supplemented media were added, immediately followed by 50 μl diluted formulations. Media only was added to columns 11 and 12 (untreated controls). After mixing, plates were incubated for 1 h at 37ºC, 5% CO₂.

Cytotoxicity Test Media was removed from the cells and replaced with 50 μl of supplemented media, followed by 50 μl of the diluted formulations or media. After mixing, the plates were incubated for 48 h at 37° C. and 5% CO₂.

### Cell Infection

After 1 h, media was removed from the cells and replaced with 50 μl of virus or media (uninfected untreated control), immediately followed by 50 μl of the formulation dilutions previously prepared. Plates were incubated for 48 h at 37° C. and 5% CO₂, and the formulations+virus were left on the cells for the entire duration of the experiment.

### Fixation and Development

After 48 h, the plates were washed with PBS, fixed for 30 mins with 4% formaldehyde, washed again with PBS, and stored in PBS at 4° C. until staining.

The cytotoxicity plates were treated with MTT to determine cell viability.

### Infectivity Readout

Cells were immunostained with immunofluorescence staining. Briefly, any residual formaldehyde was quenched with 50 mM ammonium chloride, after which cells were permeabilised (0.1% Triton X100) and stained with an antibody recognizing dengue virus envelope protein (Invitrogen MA1-27093, for DENV 1-3), pan flavivirus envelope (Millipore MAB10216, for DENV4 and ZIKV) and yellow fever E antibody 3576 (Santa Cruz, sc-58083, for YF17D). The primary antibodies were detected with an Alexa-488 conjugate secondary antibody (Life Technologies, A11001), and nuclei were stained with Hoechst. Images were acquired on a CellInsight CX5 high content platform (Thermo Scientific) using a 4X objective, and percentage infection calculated using CellInsight CX5 software (infected cells/total cells×100).

### Cytotoxicity Readout

Cytotoxicity was detected by MTT assay. Briefly, the MTT reagent (Sigma) was added to the cells for 2 h at 37° C., 5% CO₂, after which the media was removed and the precipitate solubilized with a mixture of 1:1 isopropanol:DMSO for 20 minutes. The supernatant was transferred to a clean plate and signal read at 570 nm.

### Determination of EC₅₀ Concentration

Concentration-effect data were analyzed using iterative curve fitting according to a four parameter Logistic equation (m1=maximum inhibition of infectivity, m2=EC₅₀ concentration, m3=Hill coefficient and m4=maximum infectivity; after analysis if m1>m4, then the analysis was repeated with m1 forced to equal the original m4 and m1 is missing from the table associated with the relevant graph; after the analysis if m4 was >than 10% above the highest replicate of the vehicle data then m4 was forced to the mean of the vehicle data and is evident as m4 is missing from the table associated with the relevant graph) using KaleidaGraph (v5; Synergy Software). All replicates of % infection in the absence (vehicle) or presence of indicated concentration of indicated drug were included in the analysis.

### Determination of TC₅₀ Concentration

Percentages of cytotoxicity were calculated using the following formula:

$$\% cytotoxicity = 100 - ( 100 \times \frac{Absorbance Sample}{Absorbance Untreated Control} )$$

Where the maximum cytotoxicity failed to reach the TC₅₀ value, a TC₅₀ value of >the maximal concentration tested is reported.

### Results

Table 1 displays the EC₅₀, TC₅₀, and Selectivity Index (SI(=TC₅₀/EC₅₀) for dextran sulfate, and the EC₅₀ values for the positive control inhibitors monensin.

Inhibition of all flaviviruses (i.e., the four dengue virus strains, the two Zika virus strains and the yellow fever virus strain) was evident for both dextran sulfate and positive control drugs, with EC₅₀ for dextran sulfate ranging from ˜0.03 to 0.6 mg/ml (30-600 μg/ml).

No cytotoxicity was observed at any of the dextran sulfate concentrations tested except for a low sub-TC₅₀ level at the highest concentration tested (10 mg/m1); maximally 16% cytotoxicity.

TABLE 1
EC₅₀, TC₅₀, and SI values for each virus tested against

| | Dextran sulfate | | | Monensin |
|---|---|---|---|---|
| | EC₅₀ | TC₅₀ | | EC₅₀ |
| Test Virus | (mg/ml) | (mg/ml) | SI | (nM) |
| Dengue virus 1 | 0.29 | >10 | >34 | 1.19 |
| Hawaii strain | | | | |
| Dengue virus 2 | 0.12 | >10 | >83 | 0.88 |
| New Guinea | | | | |
| C strain | | | | |
| Dengue virus 3 | 0.03 | >10 | >333 | 0.69 |
| H87 strain | | | | |
| Dengue virus 4 | 0.61 | >10 | >16 | 2.61 |
| H241 strain | | | | |
| Zika virus | 0.06 | >10 | >167 | 70.68 |
| African strain | | | | |
| Zika virus | 0.14 | >10 | >71 | 68.11 |
| Asian strain | | | | |
| Yellow Fever virus | 0.04 | >10 | >250 | 6.54 |
| 17D strain | | | | |

### Dengue Virus

Dengue is a viral infection transmitted by mosquitoes (usually the Aedes aegypti and Aedes albopictus species) and widespread in many parts of the world (Asia, Africa, the Americas, Australia and the Caribbean). Dengue infections arise from four antigenically distinct dengue virus types named DENV1, DENV2, DENV3 and DENV4. They belong to the flavivirus genus that includes the most abundant arboviral diseases of humans in term of geographical distribution, morbidity and mortality; at least 2.5 billion people are estimated to be at risk of the disease with 100-400 million dengue infections a year. Symptoms are usually mild (mild febrile illness known as ‘dengue fever’) but an estimated 500,000 cases each year are very serious and life threatening (‘severe dengue’ aka dengue haemorrhagic fever) requiring hospitalization, and about 2.5% of those affected die. According to the WHO, severe dengue is a leading cause of hospitalization and death among children and adults in Asian and Latin American countries.

There is no drug treatment or any widely available safe vaccine for dengue and dengue prevention relies on effective vector control.

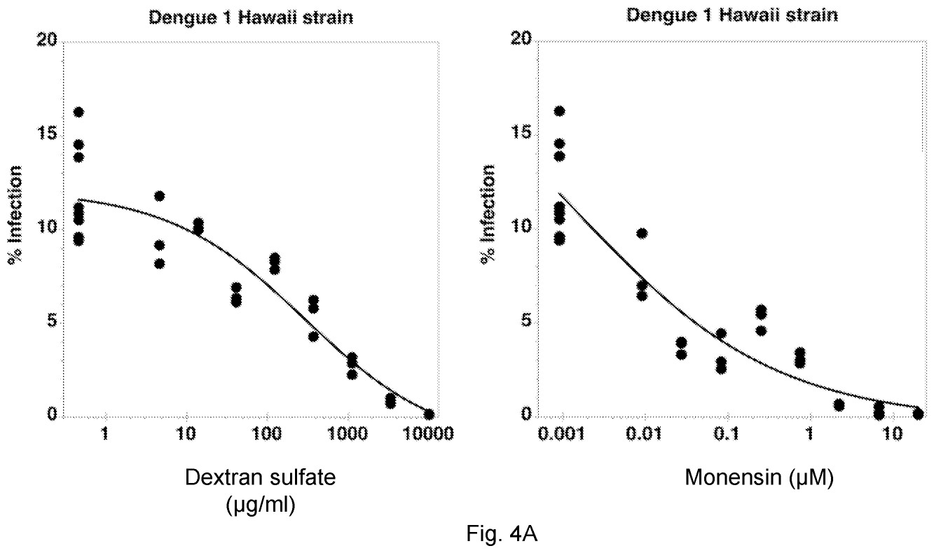

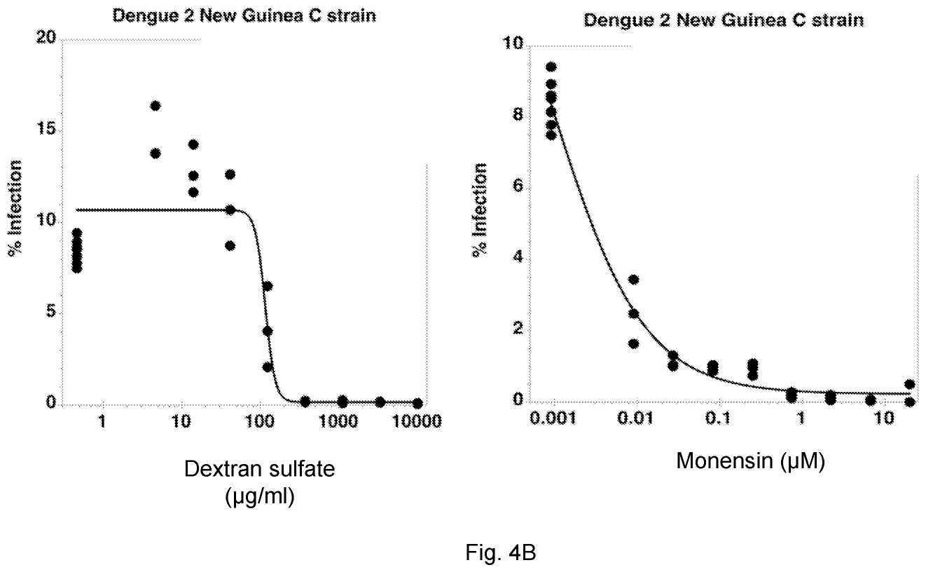

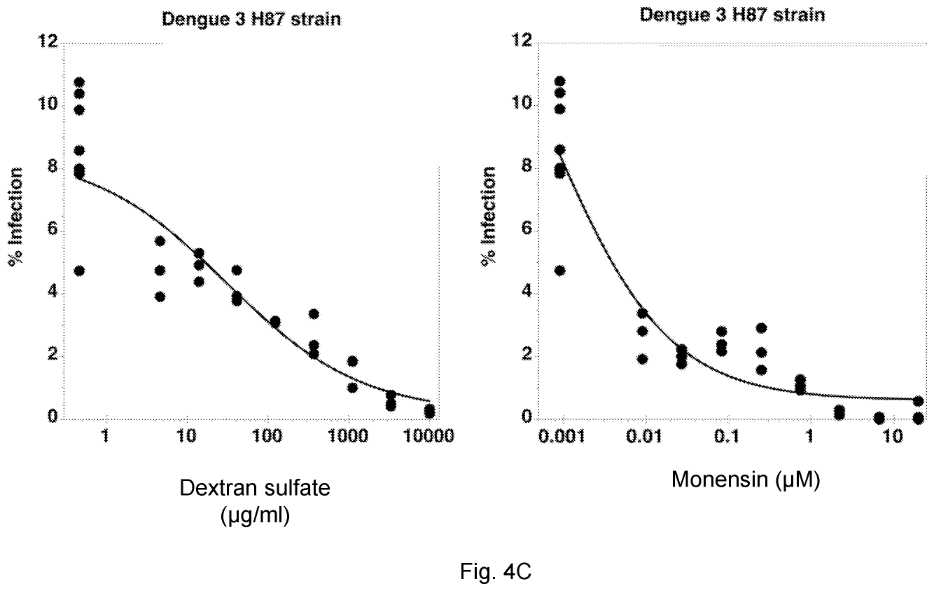

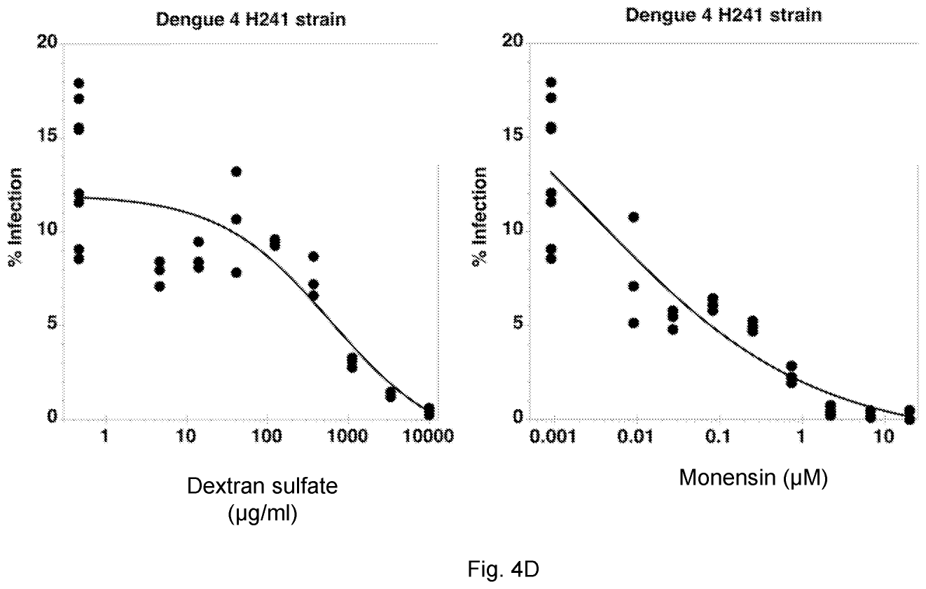

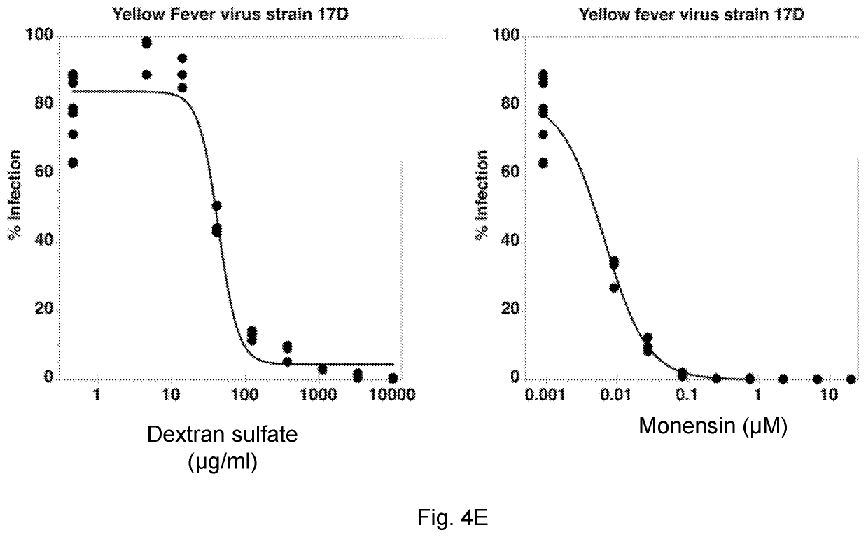

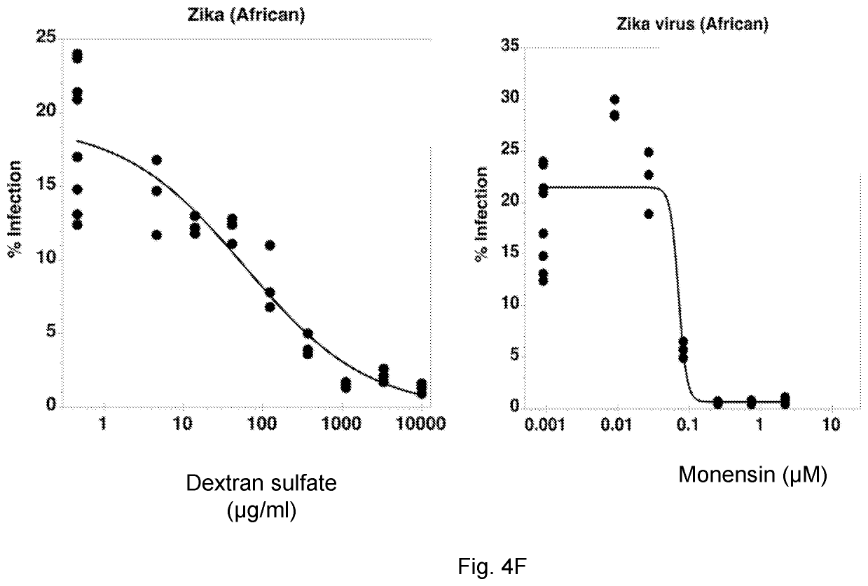

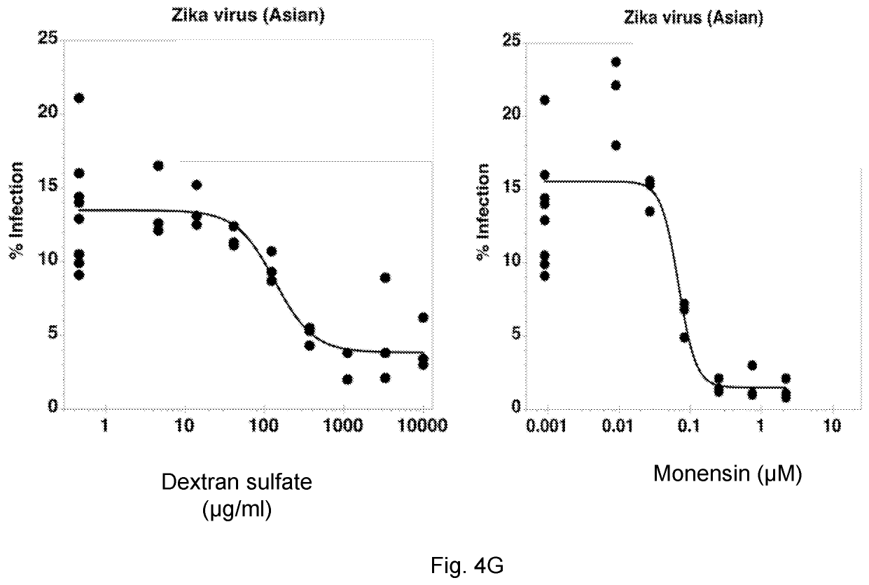

The assay assessed the ability of each of the four dengue virus types (1 to 4) to infect Huh-7 cells (human epithelial tumor cell line) and the ability of dextran sulfate to prevent the infections. Dextran sulfate displayed potent inhibition of infection by all four dengue types with IC₅₀ values of 0.6 mg/ml or lower (FIGS. 4A-4D). The assay was validated by the inhibition of infectivity from each of the four dengue variants by monensin, an ionophore with anti-viral activity.

The data with dextran sulfate supported the potential of this drug to reduce infection of humans by dengue virus.

### Yellow Fever Virus

Yellow fever is another serious arboviral diseases with the flavivirus infection spread by mosquitoes (predominantly Aedes aegypti and other Aedes species) occurring in Africa, South America, Central America and the Caribbean. Yellow fever is an acute haemorrhagic disease; the “yellow” describes the jaundice that affects some patients. Other symptoms include fever, headache, muscle pain, nausea, vomiting and fatigue which can be severe leading to death.

The assay assessed the ability of a yellow fever virus to infect Huh-7 cells (human epithelial tumor cell line) and the ability of dextran sulfate to prevent the infection. Dextran sulfate displayed potent inhibition of infection with an IC₅₀ value less than 0.05 mg/ml (FIG. 4E). The assay was validated by the inhibition of infectivity by monensin, an ionophore with anti-viral activity.

The data with dextran sulfate supported the potential of this drug to reduce infection of humans by yellow fever virus.

### Zika Virus

Like dengue virus, the flavivirus zika is transmitted by mosquitoes (again usually the Aedes aegypti and Aedes albopictus species) with outbreaks associated with the tropical and sub-tropical malarial belts of the world for example Africa, South and Central America and South and South East Asia. There are two main zika virus lineages known as the African and Asian lineage.

Whilst Zika virus disease is mild and transient for most people, the disease can have catastrophic consequences for pregnant women that transmit the virus to the fetus. This can promote miscarriage or birth defects including potentially fatal abnormally small heads (microcephaly) as well as congenital

Zika syndrome; a dysfunctional brain development disorder. In 2015-2016 a large outbreak with over 200k cases in just Brazil resulted in 8000 babies born with birth defects. It is now estimated that 5-10% of pregnancies with confirmed Zika virus infection result in birth defects, which increases to 8-15% when confirmed infection is restricted to the first trimester. In addition, there is a growing understanding that a low percentage of patients develop neurological problems, such as Guillain-Barré syndrome, neuropathy or myelitis.

There is no current medication that reduces Zika virus infection and prevention follows the precautions to reduce mosquito encounter. Infection is suspected based upon symptoms with confirmation from laboratory tests.

The assay assessed the ability of both Zika virus lineages (Asian and African) to infect Hela cells (human epithelial tumor cell line) and the ability of dextran sulfate to prevent the infection. Dextran sulfate displayed potent inhibition of infection by the two Zika virus lineages with IC₅₀ values less than 0.140 mg/ml (FIGS. 4G-4H). The assay was validated by the inhibition of infectivity from both variants by monensin, an ionophore with anti-viral activity.

The data with dextran sulfate supported the potential of this drug to reduce infection of humans by Zika virus.

### CONCLUSIONS

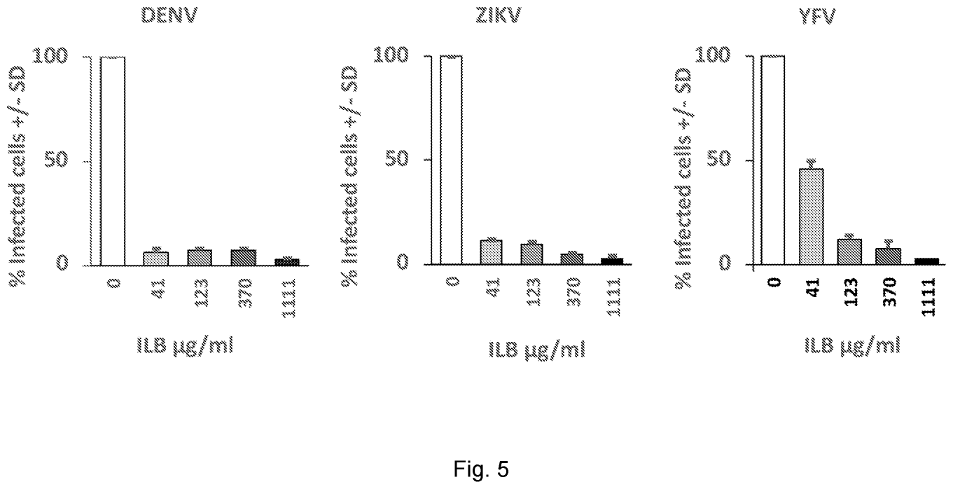

The ability of dextran sulfate to inhibit infectivity of the flaviruses dengue (four main types), Zika (two main types) and yellow fever virus was assessed, see summary of the results in FIG. 5 for Zika Asian strain (ZIKV), Dengue Hawaii strain (DENV) and YF17D.

Dextran sulfate displayed potent ability to inhibit the infectivity of all the flaviviruses tested. In addition, general cytotoxicity of dextran sulfate was also assessed in the same assays but was not evident to any concerning level in any of the assays. The data support the potential of dextran sulfate as a treatment to prevent human infection by flaviviruses that can cause devastating diseases, for which there is no current medication.

The embodiments described above are to be understood as a few illustrative examples of the present invention. It will be understood by those skilled in the art that various modifications, combinations and changes may be made to the embodiments without departing from the scope of the present invention.

In particular, different part solutions in the different embodiments can be combined in other configurations, where technically possible.

1.-18. (canceled)

19. A method for preventing, inhibiting or treating a flavivirus infection or infectious disease, the method comprises administering an effective amount of dextran sulfate, or a pharmaceutically acceptable salt thereof, having an average molecular weight equal to or below 10 000 Da and an average sulfur content of equal to or above 15% to a subject suffering from the flavivirus infection or infectious disease or having a risk of suffering from the flavivirus infection or infectious disease.

20. The method according to claim 19, wherein the flavivirus infection is caused by a flavivirus selected from the group consisting of dengue virus, Japanese encephalitis virus, Kunjin virus, Langat virus, Louping ill virus, Murray valley encephalitis virus, St. louis encephalitis virus, powassan virus, West Nile virus, Yellow fever virus and Zika virus.

21. The method according to claim 20, wherein the flavivirus infection is caused by a flavivirus selected from the group consisting of dengue virus, Japanese encephalitis virus, Kunjin virus, Louping ill virus, Murray valley encephalitis virus, St. louis encephalitis virus, powassan virus, West Nile virus, Yellow fever virus and Zika virus.

22. The method according to claim 21, wherein the flavivirus infection is caused by a flavivirus selected from the group consisting of dengue virus, Yellow fever virus and Zika virus.

23. The method according to claim 22, wherein the flavivirus infection is caused by Zika virus.

24. The method according to claim 22, wherein the flavivirus infection is caused by dengue virus.

25. The method according to claim 22, wherein the flavivirus infection is caused by Yellow fever virus.

26. The method according to claim 19, wherein the flavivirus infection is caused by a flavivirus other than Japanese encephalitis virus, West Nile virus, dengue virus, and Yellow fever virus.

27. The method according to claim 19, wherein administering an effective amount comprises systemically administering the effective amount of dextran sulfate, or the pharmaceutically acceptable salt thereof, having an average molecular weight equal to or below 10 000 Da and an average sulfur content of equal to or above 15% to the subject suffering from the flavivirus infection or infectious disease or having a risk of suffering from the flavivirus infection or infectious disease

28. The method according to claim 27, wherein administering an effective amount comprises intravenously or subcutaneously administering the effective amount of dextran sulfate, or the pharmaceutically acceptable salt thereof, having an average molecular weight equal to or below 10 000 Da and an average sulfur content of equal to or above 15% to the subject suffering from the flavivirus infection or infectious disease or having a risk of suffering from the flavivirus infection or infectious disease

29. The method according to claim 19, wherein the dextran sulfate, or the pharmaceutically acceptable salt thereof, has an average sulfur content within a range of from 15 to 20%.

30. The method according to claim 19, wherein the average molecular weight is within a range of from 2 000 to 10 000 Da.

31. The method according to claim 30, wherein the average molecular weight is within a range of from 3 000 to 10 000 Da.

32. The method according to claim 31, wherein the average molecular weight is within a range of from 3 500 to 9 500 Da.

33. The method according to claim to 32, wherein the average molecular weight is within a range of from 4 500 to 7 500 Da.

34. The method according to claim 33, wherein the average molecular weight is within a range of from 4 500 to 5 500 Da.

35. The method according to claim 19, wherein the dextran sulfate, or the pharmaceutically acceptable salt thereof, has a number average molecular weight (Mₙ) as measured by nuclear magnetic resonance (NMR) spectroscopy within a range of from 1850 to 3500 Da.

36. The method according to claim 35, wherein the dextran sulfate, or the pharmaceutically acceptable salt thereof, has a Mₙ as measured by NMR spectroscopy within a range of from 1850 to 2500 Da.

37. The method according to claim 36, wherein the dextran sulfate, or the pharmaceutically acceptable salt thereof, has a Mₙ as measured by NMR spectroscopy within a range of from 1850 to 2300 Da.

38. The method according to claim 37, wherein the dextran sulfate, or the pharmaceutically acceptable salt thereof, has a Mₙ as measured by NMR spectroscopy within a range of from 1850 to 2000 Da.

39. The method according to claim 19, wherein the dextran sulfate, or the pharmaceutically acceptable salt thereof, has an average sulfate number per glucose unit within a range of from 2.5 to 3.0.

40. The method according to claim 39, wherein the dextran sulfate, or the pharmaceutically acceptable salt thereof, has an average sulfate number per glucose unit within a range of from 2.5 to 2.8.

41. The method according to claim 40, wherein the dextran sulfate, or the pharmaceutically acceptable salt thereof, has an average sulfate number per glucose unit within a range of from 2.6 to 2.7.

42. The method according to claim 19, wherein the dextran sulfate, or the pharmaceutically acceptable salt thereof, has on average 5.1 glucose units and an average sulfate number per glucose unit of 2.6 to 2.7.

43. The method according to claim 19, wherein the dextran sulfate, or the pharmaceutically acceptable salt thereof, is formulated as an aqueous injection solution.

44. The method according to claim 19, wherein the pharmaceutically acceptable salt thereof is a sodium salt of dextran sulfate.
